(19) United States
(12) Patent Application Publication
(10) Pub. No.: US 2024/0285896 A1
(43) Pub. Date: Aug. 29, 2024

(54) EMOTION RECOGNITION INDUCTION APPARATUS, ELECTRICAL STIMULATION INTENSITY DETERMINATION METHOD AND PROGRAM

(71) Applicant: NIPPON TELEGRAPH AND TELEPHONE CORPORATION, Tokyo (JP)

(72) Inventors: Naoki HAGIYAMA, Tokyo (JP); Kana EGUCHI, Tokyo (JP); Shunichi SEKO, Tokyo (JP)

(21) Appl. No.: 18/572,502

(22) PCT Filed: Jun. 30, 2021
(86) PCT No.: PCT/JP2021/024868

§ 371 Date: Dec. 20, 2023

(51) Int. Cl. A61M 21/00; A61N 1/04

(52) U.S. Cl. CPC A61M 21/00; A61N 1/0452; A61M 2021/0072

(57) ABSTRACT

An emotion recognition inducing device includes: an electrical stimulation presentation unit that presents electrical stimulation to the body of a recognizer; a perceptual response acquisition unit that acquires feedback from the recognizer in response to the electrical stimulation; and a control unit that determines the intensity of electrical stimulation for inducing emotion recognition in the recognizer, on the basis of the feedback.

### TECHNICAL FIELD

The present invention relates to a technology for inducing emotion recognition.

### BACKGROUND ART

By applying stimulus to a recognizer who is to recognize emotion, it is possible to change the emotion being recognized by the recognizer to a specific emotion (Non Patent Literature 1).

There also is a known technique for inducing specific emotion recognition by presenting electrical stimulation to the face of the recognizer (Non Patent Literature 2).

### CITATION LIST

### Non Patent Literature

Non Patent Literature 1: Strack, Fritz, Leonard L. Martin, and Sabine Stepper. “Inhibiting and facilitating conditions of the human smile: a nonobtrusive test of the facial feedback hypothesis.” Journal of personality and social psychology 54.5 (1988): 768.
Non Patent Literature 2: Goto, Takashi, et al. “Towards Enhancing Emotional Responses to Media using Auto-Calibrating Electric Muscle Stimulation (EMS).” Proceedings of the 9th Augmented Human International Conference. 2018.

### SUMMARY OF INVENTION

### Technical Problem

By the conventional technique disclosed in Non Patent Literature 2, however, the intensity of the electrical stimulation to be presented is so high as to cause discomfort to the recognizer with the pain and stiffness from the electrical stimulation. As a result, emotion recognition cannot be induced.

The present invention has been made in view of the above aspects, and aims to provide a technology for enabling induction of emotion recognition without causing discomfort to the recognizer who is to recognize emotion.

### Solution to Problem

The technology disclosed herein is to provide an emotion recognition inducing device that includes:
an electrical stimulation presentation unit that presents electrical stimulation to the body of a recognizer;
a perceptual response acquisition unit that acquires feedback from the recognizer in response to the electrical stimulation; and
a control unit that determines the intensity of electrical stimulation for inducing emotion recognition in the recognizer, on the basis of the feedback.

### Advantageous Effects of Invention

The technology disclosed herein is to provide a technology for enabling induction of emotion recognition without causing discomfort to the recognizer who is to recognize emotion.

### BRIEF DESCRIPTION OF DRAWINGS

FIG. 1 is a configuration diagram of an emotion recognition inducing device according to an embodiment of the present invention.

FIG. 2 is a flowchart for explaining an operation of the emotion recognition inducing device.

FIG. 3 is a diagram illustrating an example hardware configuration of a device.

### DESCRIPTION OF EMBODIMENTS

The following is a description of an embodiment (the present embodiment) of the present invention, with reference to the drawings. The embodiment described below is merely an example, and embodiments to which the present invention is applied are not limited to the embodiment described below.

### Overview of an Embodiment

In the present embodiment, an emotion recognition inducing device 100 described later induces emotion recognition in a recognizer by presenting electrical stimulation to the face of the recognizer. “Inducing emotion recognition” means changing the emotion being recognized by the recognizer to another emotion, strengthen (or weaken) the recognition of the emotion being recognized by the recognizer, or the like, for example.

Specifically, the emotion recognition inducing device 100 sets the intensity of the electrical stimulation to be presented to the face of the recognizer to the limit of the intensity (subliminal) that cannot be perceived by the recognizer, to induce emotion recognition without causing discomfort to the recognizer.

Although the electrical stimulation is presented to the face of the recognizer in the above example, the electrical stimulation may be presented to any other site in the body of the recognizer as long as emotion recognition can be induced. Therefore, in the description below, the electrode installation site will be referred to as the “body” of the recognizer.

### (Device Configuration)

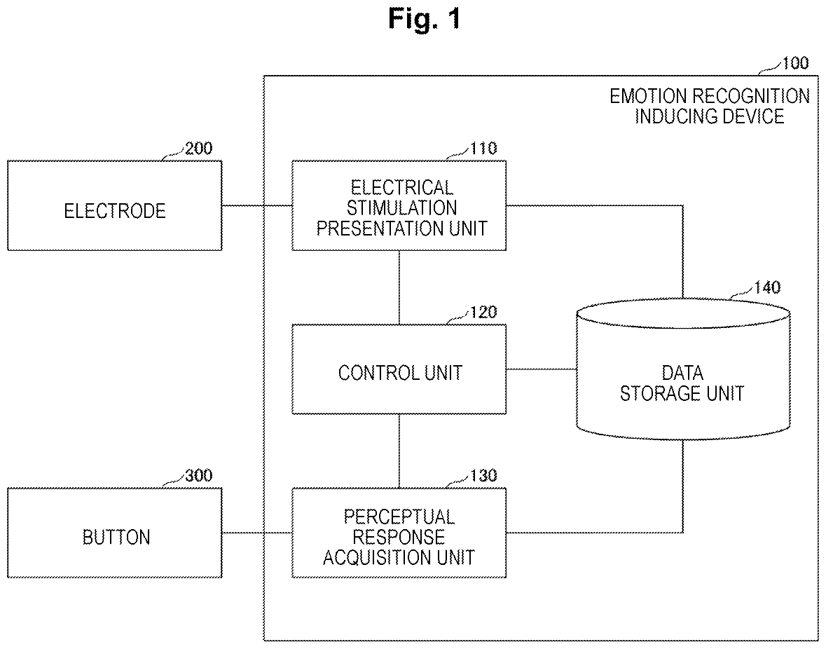

FIG. 1 illustrates an example configuration of an emotion recognition inducing device 100 according to the present embodiment. As illustrated in FIG. 1, the emotion recognition inducing device 100 according to the present embodiment includes an electrical stimulation presentation unit 110, a control unit 120, a perceptual response acquisition unit 130, and a data storage unit 140. An electrode 200 is connected to the electrical stimulation presentation unit 110. A response pushbutton switch (hereinafter referred to as the button 300) is connected to the perceptual response acquisition unit 130. An overview of each component is as described below.

The electrical stimulation presentation unit 110 presents an electrical stimulation to the body of the recognizer by causing electricity to flow in the electrode 200 installed on the body of the recognizer.

The perceptual response acquisition unit 130 is connected to the button 300, and acquires feedback (presence or absence of perception) from the recognizer on the basis of an operation of the button 300 by the recognizer, who has received the presentation of the electrical stimulation.

On the basis of the feedback from the recognizer, the control unit 120 determines the intensity of the electrical stimulation to be presented to the recognizer from the electrical stimulation presentation unit 110, and notifies the electrical stimulation presentation unit 110 of the determined intensity, to cause the electrical stimulation presentation unit 110 to present an electrical stimulation with the intensity.

The data storage unit 140 stores the intensity of the electrical stimulation presented to the recognizer, the number of trials, the feedback result, the parameters to be used in calculations, and the like.

The emotion recognition inducing device 100 may be formed with a single physical computer, may be formed with a configuration in which computers are connected to a network, or may be formed with a virtual machine in a cloud.

### (Example Operation of the Emotion Recognition Inducing Device 100)

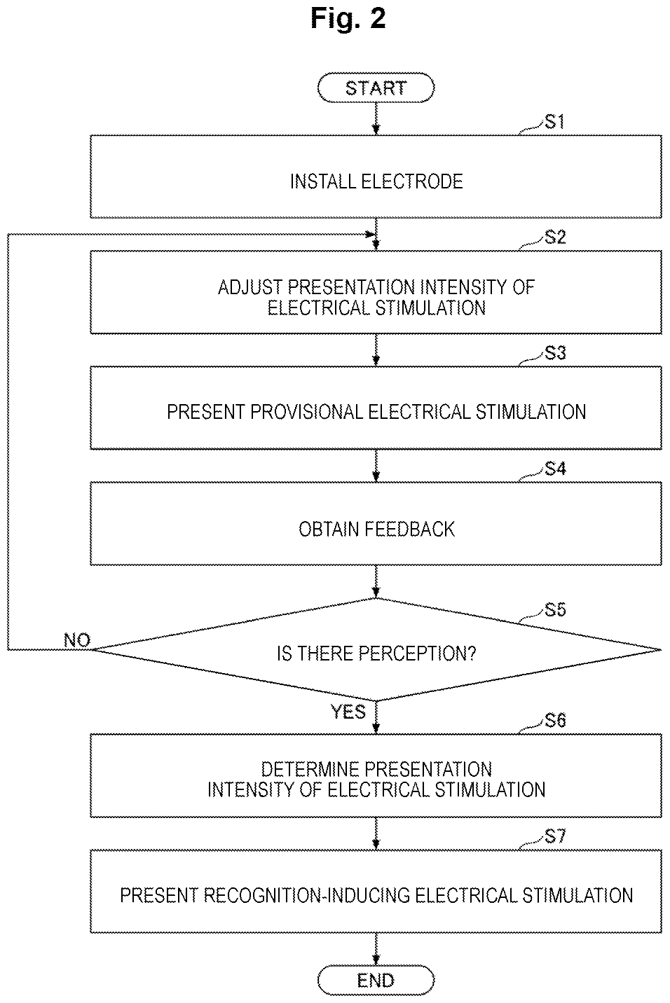

Next, an example operation of the emotion recognition inducing device 100 is described with reference to the flowchart shown in FIG. 2. Note that only S1 is not an operation to be performed by the emotion recognition inducing device 100, but an operation to be performed by a person.

### <S1>

In S1 (step 1), the electrode 200 for presenting electrical stimulation to the body of the recognizer is installed. As for the form of the electrode 200, the form as disclosed in Non Patent Literature 2 can be used, for example. However, the form of the electrode 200 is not limited to this, and may be in any form.

### <S2>

In S2, the control unit 120 adjusts the intensity of the electrical stimulation to be presented to the recognizer. That is, in the present embodiment, as illustrated in the loop from S2 to S5 in FIG. 2, a trial of presentation of the electrical stimulation is repeated while the intensity of the electrical stimulation is adjusted. The result of this repetition determines the subliminal intensity (the limit of the intensity that cannot be perceived by the recognizer).

In S2, during this repetition, the intensity of the electrical stimulation is increased stepwise with the number of trials. For example, when the number of trials is n, the presentation intensity S [mA] of the electrical stimulation is determined according to the following equation.

$$S ( n ) = 0.01 \times ( n - 1 )$$

In the above example, an arithmetic progression of 0.01 is used for the stepwise increase in the electrical stimulation intensity. However, this is merely an example, and the form of the equation is not limited to any specific one, as long as the intensity can be increased stepwise with the number of trials. For example, it is possible to use an equation in which, while n is small, the electrical stimulation intensity is increased quickly with an increase in n, and, when n is larger than a certain threshold, the rate of increase in the electrical stimulation intensity is lowered (or the electrical stimulation intensity is slowly increased).

The control unit 120 stores the value of the intensity determined (adjusted) in S2 into the data storage unit 140 together with the number n of trials at that time.

### <S3>

In S3, the control unit 120 instructs the electrical stimulation presentation unit 110 to present the electrical stimulation with the intensity S set in S2, and the electrical stimulation presentation unit 110 presents the electrical stimulation with the intensity S to the body of the recognizer, using the electrode 200.

### <S4>

In S4, the perceptual response acquisition unit 130 obtains feedback indicating whether the recognizer has perceived the electrical stimulation presented to the body of the recognizer. For example, after presented with the electrical stimulation, the recognizer is made to send a response regarding the presence or absence of perception using the button 300, so that feedback is obtained. The control unit 120 is notified of the feedback.

For example, the button 300 includes a “perception button” and a “non-perception button”. When pressing of the “perception button” is detected, it can be determined that there is perception. When pressing of the “non-perception button” is detected, it can be determined that there is no perception.

Note that the above method is an example of a feedback acquisition method. Any appropriate method other than the above may be used. For example, the recognizer may be asked a question to confirm the presence or absence of perception, and the confirmation result may be input to the perceptual response acquisition unit 130.

### <S5>

In S5, the control unit 120 determines whether there is perception by the recognizer, on the basis of the feedback obtained in S4. If there is no perception (No in S5), the operation returns to S2. If there is perception (Yes in S5), the operation moves on to S6.

### <S6>

In S6, in which the electrical stimulation is perceived by the recognizer, the control unit 120 determines the intensity S′ of the electrical stimulation at the time when the operation to induce emotion recognition in the recognizer is actually performed. The control unit 120 determines the intensity S′ to be the highest intensity among the intensities that have been obtained in the loop from S2 to S5 and cannot be perceived by the recognizer.

Specifically, if there is perception for the first time in the nth trial, the control unit 120 adopts the intensity S determined in S2 in the (n−1)th trial as the intensity S′, as shown in the following equation.

$${S}^{′} = S ( n - 1 )$$

S(n−1) may be calculated from “S(n)=0.01×(n−1)”, or may be read from the data storage unit 140.

### <S7>

The control unit 120 instructs the electrical stimulation presentation unit 110 to present an electrical stimulation with the intensity S′ to induce emotion recognition in the recognizer, and the electrical stimulation presentation unit 110 presents the electrical stimulation with the intensity S′ to the body of the recognizer, using the electrode 200.

Note that an existing technique can be used for the duration and the timing of presentation of the electrical stimulation with the intensity S′.

### (Example Hardware Configuration)

The emotion recognition inducing device 100 can be formed with a computer that is made to execute a program, for example. This computer may be a physical computer, or may be a virtual machine in a cloud.

Specifically, the emotion recognition inducing device 100 can be formed by executing a program corresponding to the processes to be performed in the emotion recognition inducing device 100, using hardware resources such as a central processing unit (CPU) and a memory included in the computer. The program is recorded in a computer-readable recording medium (such as a portable memory) so that the program can be stored and distributed. The above program can also be provided through a network such as the Internet or electronic mail.

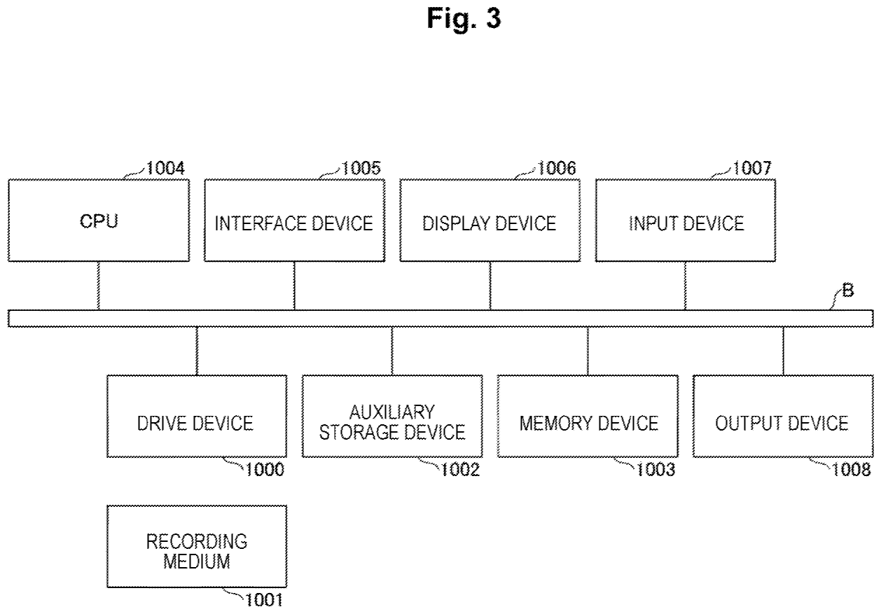

FIG. 3 is a diagram illustrating an example of the hardware configuration of the computer. The computer in FIG. 3 includes a drive device 1000, an auxiliary storage device 1002, a memory device 1003, a CPU 1004, an interface device 1005, a display device 1006, an input device 1007, and an output device 1008, which are connected to one another by a bus BS.

The program for performing the processes in the computer is provided through a recording medium 1001 such as a CD-ROM or a memory card, for example. When the recording medium 1001 storing the program is set in the drive device 1000, the program is installed into the auxiliary storage device 1002 from the recording medium 1001 via the drive device 1000. However, the program is not necessarily installed from the recording medium 1001, and may be downloaded from another computer via a network. The auxiliary storage device 1002 stores the installed program, and also stores necessary files, data, and the like.

In a case where an instruction to start the program is issued, the memory device 1003 reads and stores the program from the auxiliary storage device 1002. The CPU 1004 achieves the functions related to the emotion recognition inducing device 100, according to the program stored in the memory device 1003. The interface device 1005 is used as an interface for connection to the network, the electrode 200, the button 300, and the like. The interface device 1005 as the electrical stimulation presentation unit 110 may have a function of providing electricity to the electrode 200.

The display device 1006 displays a graphical user interface (GUI) or the like according to the program. The input device 1007 is formed with a keyboard, a mouse, buttons, a touchscreen, or the like, and is used to input various operation instructions. The output device 1008 outputs calculation results.

### Effects of the Embodiment

The technology according to the present embodiment described above makes it possible to induce emotion recognition without causing discomfort to the recognizer who is to recognize emotion.

### SUMMARY OF THE EMBODIMENT

This specification discloses at least the emotion recognition inducing device, the electrical stimulation intensity determining method, and the program described in the following items.

### (Item 1)

An emotion recognition inducing device including:
an electrical stimulation presentation unit that presents electrical stimulation to a body of a recognizer;
a perceptual response acquisition unit that acquires feedback from the recognizer in response to the electrical stimulation; and
a control unit that determines an intensity of electrical stimulation for inducing emotion recognition in the recognizer, on the basis of the feedback.

### (Item 2)

The emotion recognition inducing device of item 1, in which
the control unit determines the intensity of the electrical stimulation for inducing emotion recognition in the recognizer, by increasing stepwise the intensity of the electrical stimulation to be presented by the electrical stimulation presentation unit while the recognizer does not perceive the electrical stimulation.

### (Item 3)

The emotion recognition inducing device of item 1 or 2, in which
the control unit determines that the highest intensity among intensities of electrical stimulation the recognizer is unable to perceive is the intensity of the electrical stimulation for inducing emotion recognition in the recognizer.

### (Item 4)

An electrical stimulation intensity determining method implemented by a computer,
the electrical stimulation intensity determining method including:
a step of presenting electrical stimulation to a body of a recognizer;
a step of acquiring feedback from the recognizer in response to the electrical stimulation; and
a step of determining an intensity of electrical stimulation for inducing emotion recognition in the recognizer, on the basis of the feedback.

### (Item 5)

A program for causing a computer to function as each component in the emotion recognition inducing device according to any one of Items 1 to 3.

Although the present embodiment has been described so far, the present invention is not limited to such a specific embodiment, and various modifications and changes can be made to it within the scope of the present invention disclosed in the claims.

Reference Signs List

| | |
|---|---|
| 100 | emotion recognition inducing device |
| 110 | electrical stimulation presentation unit |
| 120 | control unit |
| 130 | perceptual response acquisition unit |
| 140 | data storage unit |
| 200 | electrode |
| 300 | button |
| 1000 | drive device |
| 1001 | recording medium |
| 1002 | auxiliary storage device |
| 1003 | memory device |
| 1004 | CPU |
| 1005 | interface device |
| 1006 | display device |
| 1007 | input device |

1. An emotion recognition inducing device comprising:
a memory; and
a processor configured to:
present electrical stimulation to a body of a recognizer;
acquire feedback from the recognizer in response to the electrical stimulation; and
determine an intensity of electrical stimulation for inducing emotion recognition in the recognizer, on a basis of the feedback.

2. The emotion recognition inducing device according to claim 1, wherein
the processor determines the intensity of the electrical stimulation for inducing emotion recognition in the recognizer, by increasing stepwise the intensity of the electrical stimulation to be presented while the recognizer does not perceive the electrical stimulation.

3. The emotion recognition inducing device according to claim 1, wherein
the processor determines that a highest intensity among intensities of electrical stimulation that cannot be perceived by the recognizer is the intensity of the electrical stimulation for inducing emotion recognition in the recognizer.

4. An electrical stimulation intensity determining method executed by a computer, the electrical stimulation intensity determining method comprising:
presenting electrical stimulation to a body of a recognizer;
acquiring feedback from the recognizer in response to the electrical stimulation; and
determining an intensity of electrical stimulation for inducing emotion recognition in the recognizer, on a basis of the feedback.

5. (canceled)

6. A non-transitory computer-readable recording medium having computer-readable instructions stored thereon, which, when executed, cause a computer to execute a method, the method comprising:
presenting electrical stimulation to a body of a recognizer;
acquiring feedback from the recognizer in response to the electrical stimulation; and
determining an intensity of electrical stimulation for inducing emotion recognition in the recognizer, on a basis of the feedback.
